(19) United States
(12) Patent Application Publication
(10) Pub. No.: US 2025/0073368 A1
(43) Pub. Date: Mar. 6, 2025

(54) BLUETOOTH SPEAKER TABLE LAMP WITH ESSENTIAL OIL AROMATHERAPY

(71) Applicant: Shenzhen Jiewei auspicious industrial Co., LTD, Shenzhen (CN)

(72) Inventor: Guohong XU, Wenzhou (CN)

(73) Assignee: Shenzhen Jiewei auspicious industrial Co., LTD, Shenzhen (CN)

(21) Appl. No.: 18/527,300

(22) Filed: Dec. 3, 2023

(30) Foreign Application Priority Data
Aug. 28, 2023 (CN) 2023223204525

(51) Int. Cl. A61L 9/14; A61M 11/02; F21V 33/00; H04R 1/02; H04R 3/00

(52) U.S. Cl. CPC A61L 9/14; A61M 11/02; F21V 33/0024; F21V 33/0056; H04R 1/028; H04R 3/00; A61M 2205/07; H04R 2201/028; H04R 2420/07; H04R 2420/09

(57) ABSTRACT

The present utility model discloses a Bluetooth speaker table lamp with essential oil aromatherapy, including a base, an aromatherapy mechanism arranged on the base, a lamp panel arranged around the aromatherapy mechanism, a lampshade sleeved on the lamp panel and abutting against a top of the base, and an aromatherapy control board arranged at a top of the aromatherapy mechanism, where the aromatherapy mechanism includes an essential oil bottle, a microporous tube B communicating with the essential oil bottle, a silicone pad of the essential oil bottle that is arranged on a bottle mouth of the essential oil bottle, an aromatherapy chamber detachably connected to the bottle mouth of the essential oil bottle, an air pump motor arranged beside the essential oil bottle, and a microporous tube A with one end communicating with the air pump motor.

### CROSS-REFERENCE TO RELATED APPLICATIONS

The application claims priority to Chinese patent application No. 2023223204525, filed on Aug. 28, 2023, the entire contents of which are incorporated herein by reference.

### TECHNICAL FIELD

The present utility model relates to the technical field of table lamps, in particular to a Bluetooth speaker table lamp with essential oil aromatherapy.

### BACKGROUND

As a commonly used lighting tool in people's life, the table lamp is highly favored by the public because of its simple and practical structure. With the improvement of people's living standards, the requirements for the quality of life are constantly improving. With the development of the times, people also pay more and more attention to sleep health. Table lamps are one of the most common products placed at the bedside by people. Table lamps in the prior art can achieve good lighting, but have few functions and only have a single lighting function. It is rare to combine the function of a table lamp having an aromatherapy function with a Bluetooth speaker on the market. Therefore, this technology is developed to help people add the function of healthy sleep to the table lamp.

### SUMMARY

In view of the problems existing in the prior art, the present utility model provides a Bluetooth speaker table lamp with essential oil aromatherapy.

To achieve the above objective, the present utility model adopts the following technical solution:

The present utility model provides a Bluetooth speaker table lamp with essential oil aromatherapy, including a base, an aromatherapy mechanism arranged on the base, a lamp panel arranged around the aromatherapy mechanism, a lampshade sleeved on the lamp panel and abutting against a top of the base, and an aromatherapy control board arranged at a top of the aromatherapy mechanism,
where the aromatherapy mechanism includes an essential oil bottle, a microporous tube B communicating with the essential oil bottle, a silicone pad of the essential oil bottle that is arranged on a bottle mouth of the essential oil bottle, an aromatherapy chamber detachably connected to the bottle mouth of the essential oil bottle, an air pump motor arranged beside the essential oil bottle, and a microporous tube A with one end communicating with the air pump motor; the other end of the microporous tube A penetrates through the aromatherapy chamber and is opposite to a top of the microporous tube B penetrating through the aromatherapy chamber;
a main control board, a speaker cavity, and a speaker arranged on the speaker cavity are arranged in the base; and the main control board is provided with a Bluetooth module.

Preferably, the base is further provided with control buttons and interfaces; the control buttons are opposite to the speaker cavity, and the speaker cavity is arranged above the main control board; and the interfaces include a TYPE-C interface and a USB interface.

Preferably, the TYPE-C interface is a power input port configured to supply power to the main control board, the lamp panel, and the air pump motor of the aromatherapy mechanism.

Preferably, the aromatherapy mechanism further includes a spacer plate, a mounting sleeve, and an aromatherapy chamber cover; the spacer plate is arranged between the essential oil bottle and the air pump motor; the mounting sleeve is sleeved on the essential oil bottle and the air pump motor; the aromatherapy chamber cover has one side fixed to the aromatherapy chamber and provided with an atomization hole, and the other side provided with an aromatherapy control board mounting hole, and an atomization hole cover arranged below the atomization hole is further arranged in the aromatherapy chamber; the aromatherapy control board is arranged at the aromatherapy control board mounting hole and is opposite to the air pump motor below; a power wire is further arranged below the air pump motor; and the lamp panel is arranged around the mounting sleeve.

Preferably, the microporous tube A and the microporous tube B are arranged at 90°.

Preferably, the aromatherapy chamber is detachably connected to the essential oil bottle through threads.

Preferably, the main control board is further provided with a main control circuit, an audio power amplifier circuit, a button switch circuit, a storage circuit, an interface circuit, and a lamp panel circuit; and
corresponding ports of the main control circuit are electrically connected to corresponding ports of the audio power amplifier circuit, the button switch circuit, the storage circuit, the interface circuit, and the Bluetooth module.

Preferably, the main control circuit includes a main control chip CA51F005 and a peripheral circuit thereof.

Preferably, the audio power amplifier circuit includes an audio power amplifier chip XS9110A and a peripheral circuit thereof; and corresponding ports of the audio power amplifier chip XS9110A are electrically connected to corresponding ports of the main control chip CA51F005.

Preferably, the Bluetooth module includes a Bluetooth circuit, the Bluetooth circuit including a Bluetooth chip and a peripheral circuit thereof; and corresponding ports of the Bluetooth chip are electrically connected to the corresponding ports of the main control chip CA51F005.

With the technical solution of the present utility model, the following beneficial effects are achieved:

The present utility model creates a three-in-one function, which combines functions of aromatherapy, table lamp lighting, and a Bluetooth speaker together, such that consumers have one more choice. The aromatherapy may help people soothe the nerves and help sleep. Moreover, both the manufacturing costs of products and the purchase costs of the consumers are saved. The air pump motor causes a high-speed airflow to be formed through the microporous tube A and blows it towards the top of another microporous tube B connected to the essential oil bottle, such that a negative pressure is formed in the microporous tube B to drive essential oil in the essential oil bottle to flow out from the top of the microporous tube B. When flowing out from the top of the microporous tube B, the essential oil is blown away by the high-speed airflow of the microporous tube A, thereby achieving an atomization effect.

### BRIEF DESCRIPTION OF DRAWINGS

FIG. 1 is a first schematic structural diagram of the present utility model;

FIG. 2 is a second schematic structural diagram of the present utility model;

FIG. 3 is a schematic diagram of internal structures of an aromatherapy mechanism, a lamp panel, and a base in the present utility model;

FIG. 4 is a first exploded view of an aromatherapy mechanism in the present utility model;

FIG. 5 is a second exploded view of an aromatherapy mechanism in the present utility model;

FIG. 6 is a third exploded view of an aromatherapy mechanism in the present utility model;

FIG. 7 is a schematic structural diagram of a microporous tube A and a microporous tube B of an aromatherapy mechanism in the present utility model;

FIG. 8 is a schematic diagram of a main control circuit in the present utility model;

FIG. 9 is a schematic circuit diagram of a Bluetooth module in the present utility model;

FIG. 10 is a schematic diagram of an audio power amplifier circuit in the present utility model;

FIG. 11 is a schematic diagram of a storage circuit in the present utility model;

FIG. 12 is a schematic diagram of a lamp panel circuit in the present utility model;

FIG. 13A and FIG. 13B is a schematic diagram of an interface circuit in the present utility model;

FIG. 14 is a schematic diagram of a button switch circuit in the present utility model; and

FIG. 15 is a function control diagram of the present utility model.

### DETAILED DESCRIPTION OF THE EMBODIMENTS

Embodiments of the present utility model are described in detail below. Examples of the embodiments are shown in the accompanying drawings, in which the same or similar reference numerals represent the same or similar elements or elements with the same or similar functions throughout. The embodiments described below with reference to the accompanying drawings are exemplary, are intended to explain the present utility model, and should not be construed as a limitation to the present utility model.

In the description of the present utility model, it should be understood that the orientations or positional relationships indicated by the terms “center”, “longitudinal”, “transverse”, “length”, “width”, “thickness”, “upper”, “lower”, “front”, “back”, “left”, “right”, “vertical”, “horizontal”, “top”, “bottom”, “inside”, “outside”, “clockwise”, “anticlockwise”, etc. are based on the orientations or positional relationships shown in the accompanying drawings, are only for the convenience of describing the present utility model and simplifying the description rather than indicating or implying that the apparatus or element referred to must have a particular orientation or be constructed and operated in a particular orientation, and therefore should not be construed as a limitation to the present utility model.

In addition, the terms “first” and “second” are only used for descriptive purposes, and cannot be construed as indicating or implying relative importance or implying the number of technical features indicated. Thus, the features defined with “first” and “second” may explicitly or implicitly include one or more of the features. In the description of the present utility model, “a plurality of” means two or more, unless otherwise expressly and specifically defined.

In the present utility model, unless otherwise explicitly specified and defined, the terms “mounted”. “connected”, “connection”, “fixed”, etc. should be understood in a broad sense, for example, it may be a fixed connection, a detachable connection, or an integrated connection; it may be a mechanical connection or an electrical connection; and it may be being directly connected, being indirectly connected via an intermediate medium, or communication between interiors of two elements. Those of ordinary skill in the art may understand specific meanings of the above terms in the present utility model according to specific circumstances.

In the present utility model, the state that the first feature is “over” or “under” the second feature may include a state that the first and second features are in direct contact with each other, or it may include a state that the first and second features are not in direct contact with each other and are in contact with each other via another feature between them, unless otherwise expressly specified and defined.

Moreover, the state that the first feature is “over”, “above”, and “on” the second feature includes a state that the first feature is right above and obliquely above the second feature, or only indicates that the horizontal height of the first feature is greater than the horizontal height of the second feature. The state that the first feature is “under”. “below”, and “beneath” the second feature may include a state that the first feature is right below or obliquely below the second feature, or only indicates that the horizontal height of the first feature is less than the horizontal height of the second feature.

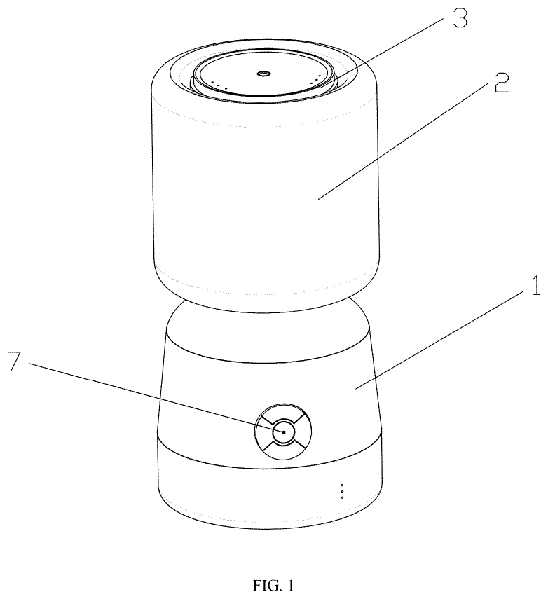

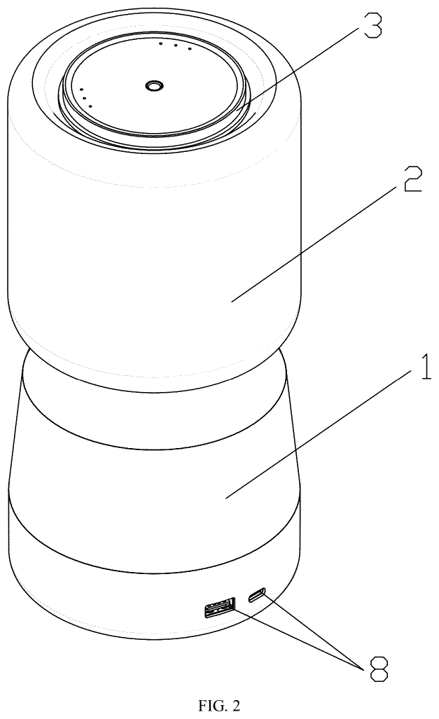

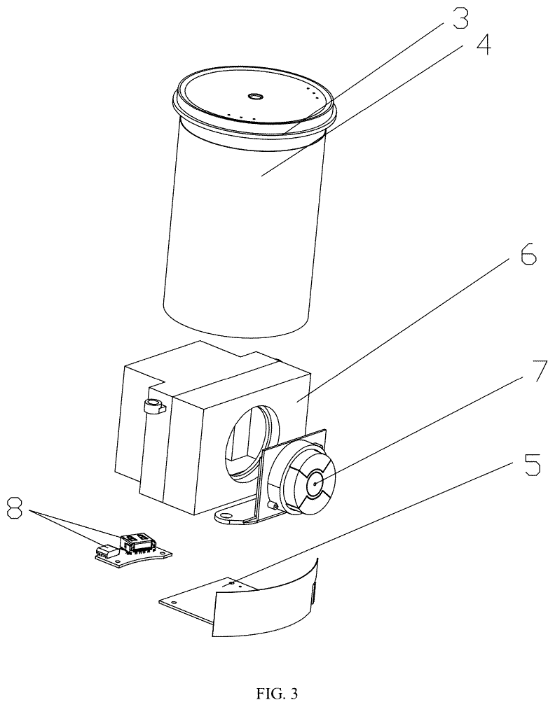

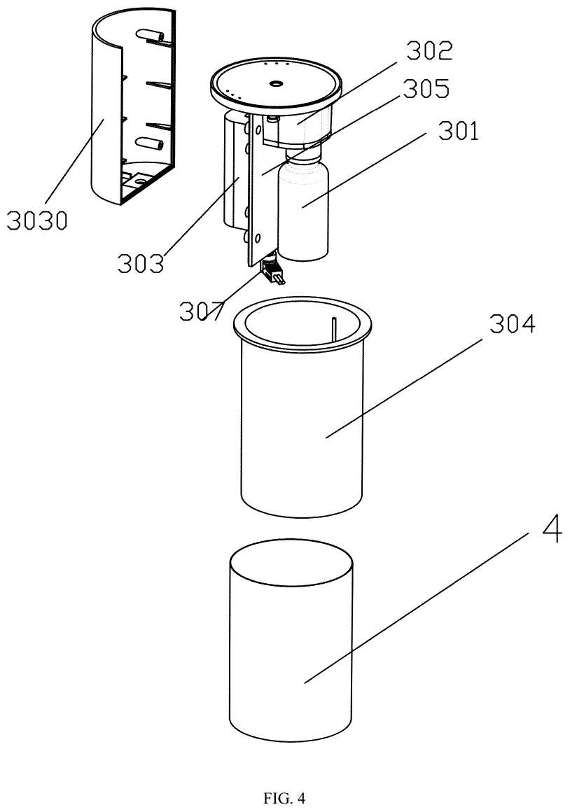

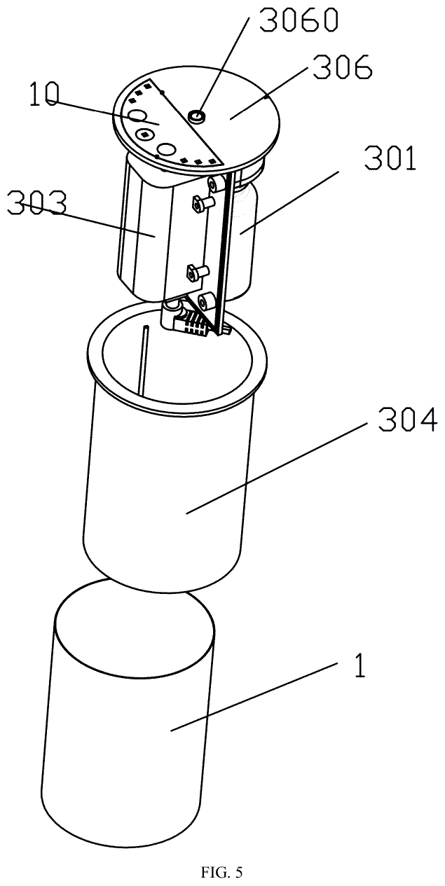

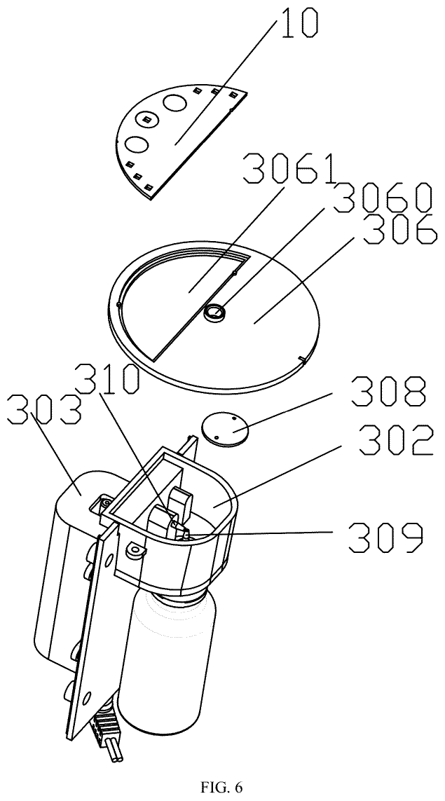

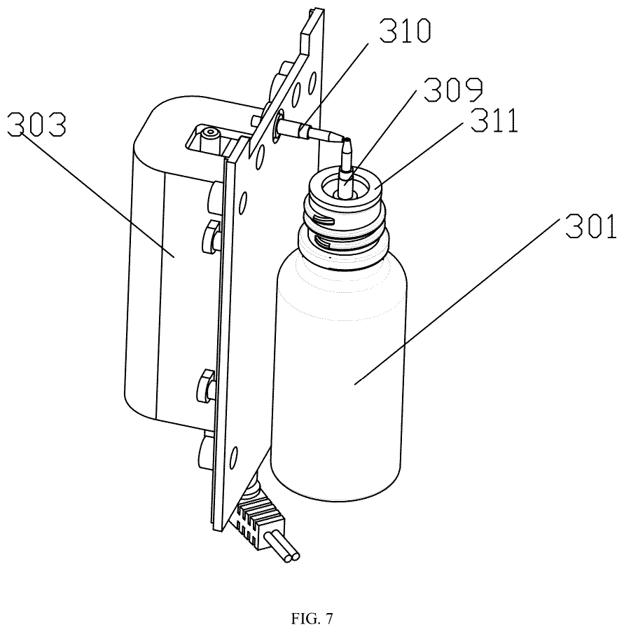

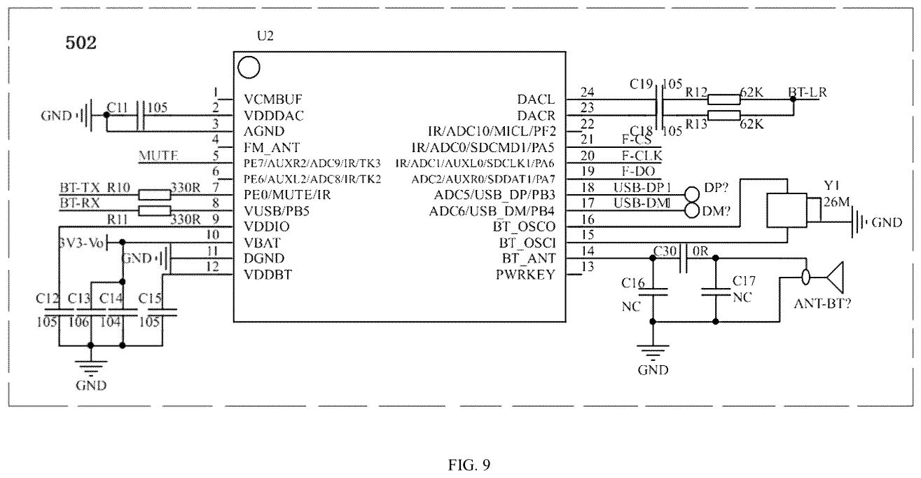

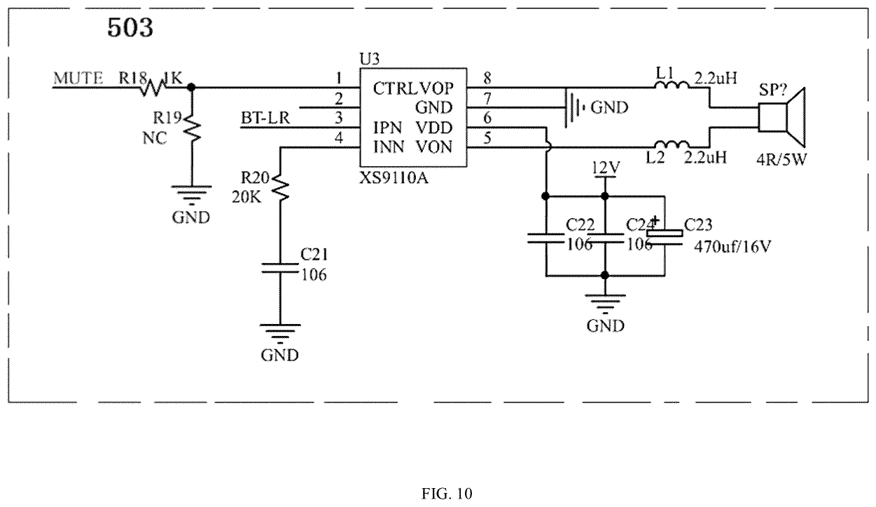

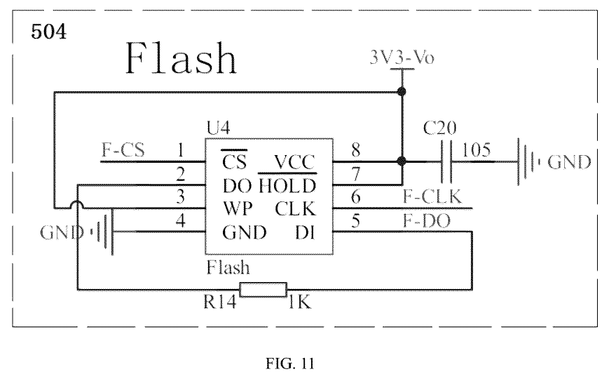

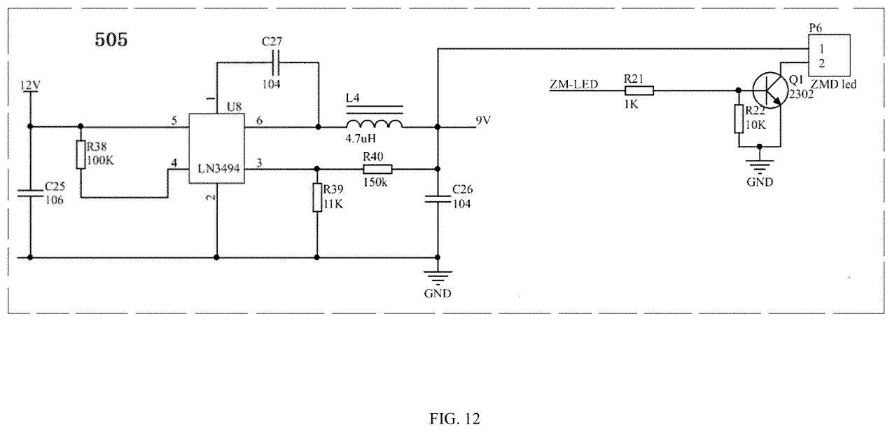

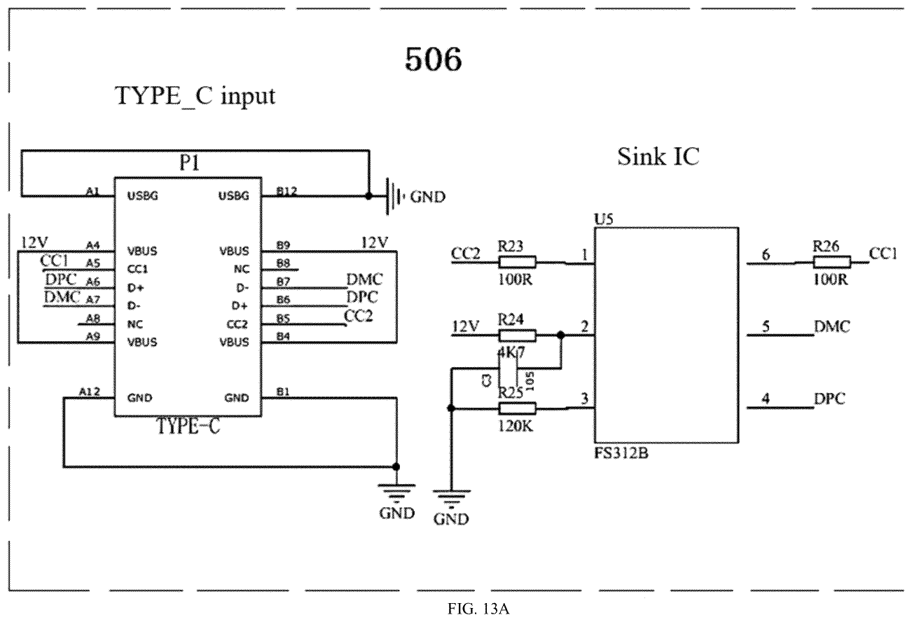

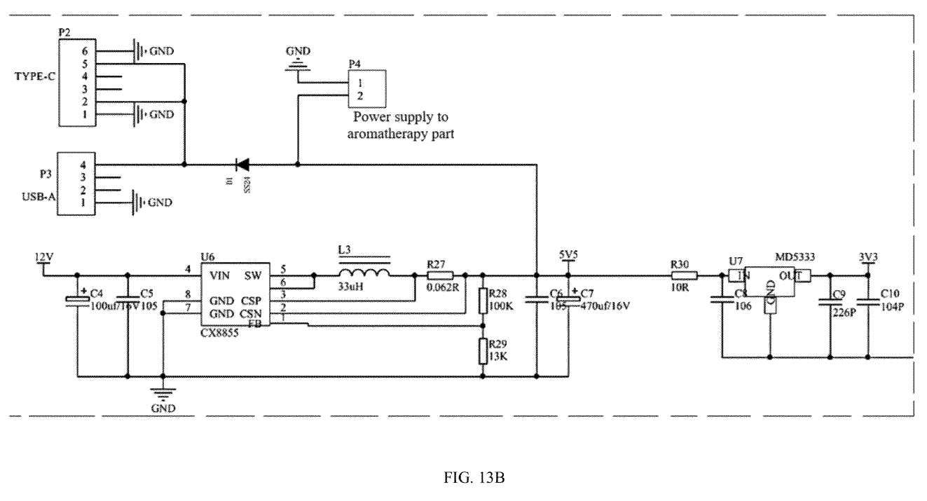

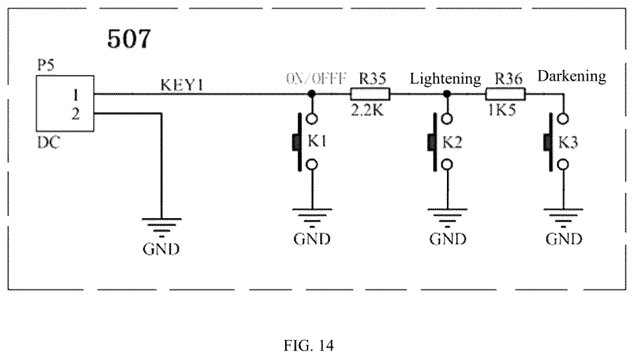

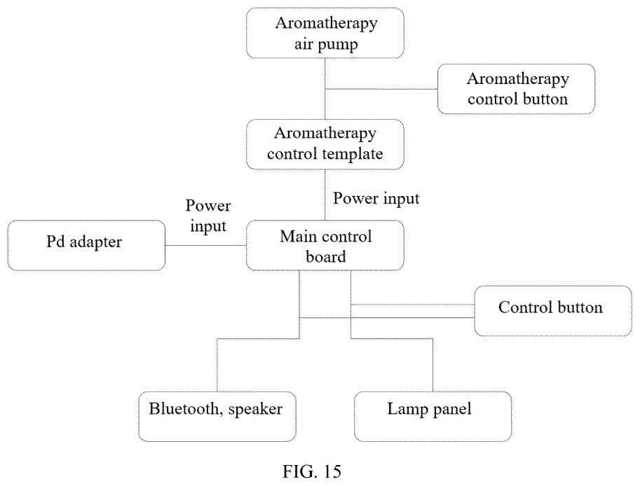

Referring to FIG. 1 to FIG. 15, the present utility model provides a Bluetooth speaker table lamp with essential oil aromatherapy, including a base 1, an aromatherapy mechanism arranged on the base 1, a lamp panel 4 arranged around the aromatherapy mechanism 3, a lampshade 2 sleeved on the lamp panel 4 and abutting against a top of the base, and an aromatherapy control board 10 arranged at a top of the aromatherapy mechanism 3. The aromatherapy control board 10 is used for the independent aromatherapy mechanism 3 to work. The aromatherapy control board 10 is further mounted with an aromatherapy control button for controlling an air pump motor 303 of the aromatherapy mechanism 3 to start or stop. The aromatherapy control board 10 is further provided with a gear indicator light circuit, a timing indicator light circuit, and a working indicator light circuit. The lampshade 2 is a semitransparent lampshade wrapping the lamp panel 4. A speaker cavity 6 is connected to a control mainboard 5 through two wires. The lamp panel 4 is connected to the control mainboard 5 through two wires. The aromatherapy mechanism 3 includes an essential oil bottle 301, a microporous tube B 309 communicating with the essential oil bottle 301, a silicone pad 311 of the essential oil bottle that is arranged on a bottle mouth of the essential oil bottle 301, an aromatherapy chamber 302 detachably connected to the bottle mouth of the essential oil bottle 301, the air pump motor 303 arranged beside the essential oil bottle 301, and a microporous tube A 310 with one end communicating with the air pump motor 303. The microporous tube A 310 and the microporous tube B 309 are set as capillary tubes. The other end of the microporous tube A 310 penetrates through the aromatherapy chamber 302 and is opposite to a top of the microporous tube B 309 penetrating through the aromatherapy chamber 302. The aromatherapy mechanism 3 further includes a spacer plate 305, a mounting sleeve 304, and an aromatherapy chamber cover 306. The spacer plate 305 is arranged between the essential oil bottle 301 and the air pump motor 303. The mounting sleeve 304 is sleeved on the essential oil bottle 301 and the air pump motor 303. The aromatherapy chamber cover 306 has one side fixed to the aromatherapy chamber 302 and provided with an atomization hole 3060, and the other side provided with an aromatherapy control board mounting hole 3061, and an atomization hole cover 308 arranged below the atomization hole 3060 is further arranged in the aromatherapy chamber 302. The aromatherapy control board 10 is arranged at the aromatherapy control board mounting hole 3061 and is opposite to the air pump motor 303 below. A power wire 307 is further arranged below the air pump motor 303, and the air pump motor 303 is provided with an air pump motor cover 3030 for covering the air pump motor to prevent dust. The lamp panel 4 is arranged around the mounting sleeve 304. The microporous tube A 310 and the microporous tube B 309 are arranged at 90°, which is convenient to blow away an airflow of essential oil in the essential oil bottle 301.

Further, the aromatherapy chamber 302 is detachably connected to the essential oil bottle 301 through threads, which facilitates the dismounting. The aromatherapy chamber 302 is configured to store an aromatherapy material. The essential oil bottle 301 is configured to store the essential oil. The aromatherapy chamber 302 is pressed on the silicone pad 311 of the essential oil bottle. The microporous tube B 309 and the microporous tube A 310 are connected to the essential oil bottle 301 and the air pump motor 303, respectively. The principle is as follows: the air pump motor 303 causes a high-speed airflow to be formed through the microporous tube A 310 and blows it towards the top of another microporous tube B 309 connected to the essential oil bottle 301, such that a negative pressure is formed in the microporous tube B 309 to drive the essential oil in the essential oil bottle 301 to flow out from the top of the microporous tube B 309. When flowing out from the top of the microporous tube B 309, the essential oil is blown away by the high-speed airflow of the microporous tube A 310, thereby achieving an atomization effect. A main control board 5, the speaker cavity 6, and a speaker arranged on the speaker cavity 6 are arranged in the base 1. The main control board 5 is provided with a Bluetooth module 502, which can be connected to an external intelligent device through Bluetooth for playing music. The main control board 5 is arranged at a bottom of the base 1. The base 1 is further provided with control buttons 7 and interfaces 8. The control buttons 7 are configured to control lamplight of the lamp panel 4 and functions of a Bluetooth speaker. The control buttons 7 are opposite to the speaker cavity 6. The speaker cavity 6 is arranged above the main control board 5. The interfaces 8 are arranged on one side of the main control board 5. The interfaces 8 include a TYPE-C interface and a USB interface. The TYPE-C interface is a power input port configured to supply power to the main control board 5, the lamp panel 4, and the air pump motor 303 of the aromatherapy mechanism 3.

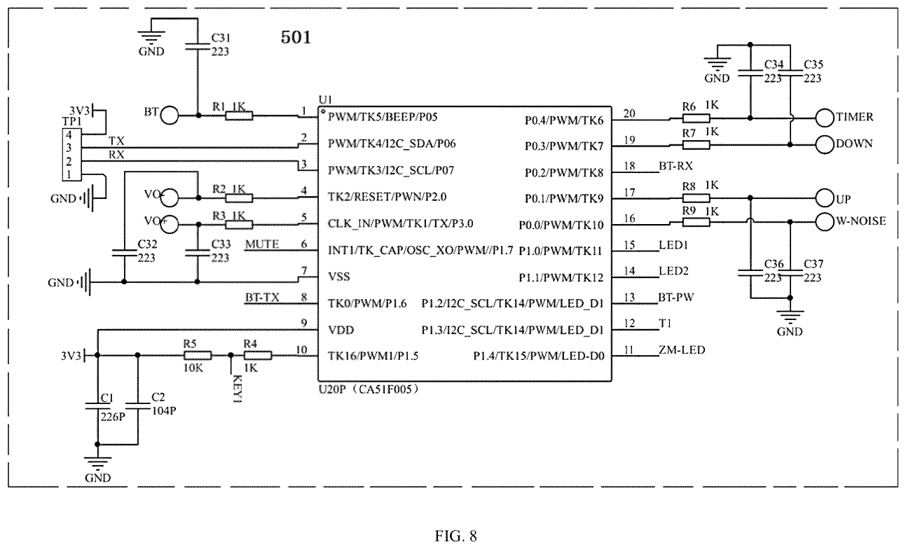

Referring to FIG. 8 to FIG. 15, the main control board 5 is further provided with a main control circuit 501, an audio power amplifier circuit 503, a button switch circuit 507, a storage circuit 504, an interface circuit 506, and a lamp panel circuit 505; corresponding ports of the main control circuit 501 are electrically connected to corresponding ports of the audio power amplifier circuit 503, the button switch circuit 507, the storage circuit 504, the interface circuit 506, and the Bluetooth module 502: corresponding ports of the button switch circuit 507 are electrically connected to corresponding ports of the control buttons 7, and corresponding ports of the audio power amplifier circuit 503 are electrically connected to corresponding ports of the speaker; corresponding ports of the interface circuit 506 are electrically connected to corresponding ports of the TYPE-C interface and the USB interface; corresponding ports of the lamp panel 4 circuit 505 are electrically connected to corresponding ports of the lamp panel; and the main control circuit 501 includes a main control chip CA51F005 and a peripheral circuit thereof. The audio power amplifier circuit 503 includes an audio power amplifier chip XS9110A and a peripheral circuit thereof; and corresponding ports of the audio power amplifier chip XS9110A are electrically connected to corresponding ports of the main control chip CA51F005. The Bluetooth module 502 includes a Bluetooth circuit, the Bluetooth circuit including a Bluetooth chip and a peripheral circuit thereof; and corresponding ports of the Bluetooth chip are electrically connected to the corresponding ports of the main control chip CA51F005.

The working principle of the present utility model is as follows:

The control buttons 7 are pressed to open the lamp panel 3 and turn on the Bluetooth module and the speaker; the Bluetooth module communicates with a handheld device; music in the handheld device is turned on and played through the speaker; then the aromatherapy control button on the aromatherapy control board 10 is turned on to control the air pump motor 303 to start; the air pump motor 303 causes the high-speed airflow to be formed through the microporous tube A 310 and blows it towards the top of another microporous tube B 309 connected to the essential oil bottle 301, such that the negative pressure is formed in the microporous tube B 309 to drive the essential oil in the essential oil bottle 301 to flow out from the top of the microporous tube B 309; and when flowing out from the top of the microporous tube B 309, the essential oil is blown away by the high-speed airflow of the microporous tube A 310, thereby achieving the atomization effect.

The present utility model creates a three-in-one function, which combines functions of aromatherapy, table lamp lighting, and the Bluetooth speaker together, such that consumers have one more choice. The aromatherapy may help people soothe the nerves and help sleep. Moreover, both the manufacturing costs of products and the purchase costs of the consumers are saved.

The above are only preferred embodiments of the present utility model, rather than limiting the scope of patent of the present utility model. Any equivalent structure transformations made using the content of the specification and the accompanying drawings of the present utility model in a utility model conception of the present utility model, directly or indirectly applied in other related technical fields, are all included within the scope of patent protection of the present utility model.

What is claimed is:

1. A Bluetooth speaker table lamp with essential oil aromatherapy, comprising a base, an aromatherapy mechanism arranged on the base, a lamp panel arranged around the aromatherapy mechanism, a lampshade sleeved on the lamp panel and abutting against a top of the base, and an aromatherapy control board arranged at a top of the aromatherapy mechanism,
wherein the aromatherapy mechanism comprises an essential oil bottle, a microporous tube B communicating with the essential oil bottle, a silicone pad of the essential oil bottle that is arranged on a bottle mouth of the essential oil bottle, an aromatherapy chamber detachably connected to the bottle mouth of the essential oil bottle, an air pump motor arranged beside the essential oil bottle, and a microporous tube A with one end communicating with the air pump motor; the other end of the microporous tube A penetrates through the aromatherapy chamber and is opposite to a top of the microporous tube B penetrating through the aromatherapy chamber;
a main control board, a speaker cavity, and a speaker arranged on the speaker cavity are arranged in the base; and the main control board is provided with a Bluetooth module.

2. The Bluetooth speaker table lamp with essential oil aromatherapy according to claim 1, wherein the base is further provided with control buttons and interfaces; the control buttons are opposite to the speaker cavity, and the speaker cavity is arranged above the main control board; and the interfaces comprise a TYPE-C interface and a USB interface.

3. The Bluetooth speaker table lamp with essential oil aromatherapy according to claim 2, wherein the TYPE-C interface is a power input port configured to supply power to the main control board, the lamp panel, and the air pump motor of the aromatherapy mechanism.

4. The Bluetooth speaker table lamp with essential oil aromatherapy according to claim 1, wherein the aromatherapy mechanism further comprises a spacer plate, a mounting sleeve, and an aromatherapy chamber cover; the spacer plate is arranged between the essential oil bottle and the air pump motor; the mounting sleeve is sleeved on the essential oil bottle and the air pump motor; the aromatherapy chamber cover has one side fixed to the aromatherapy chamber and provided with an atomization hole, and the other side provided with an aromatherapy control board mounting hole, and an atomization hole cover arranged below the atomization hole is further arranged in the aromatherapy chamber; the aromatherapy control board is arranged at the aromatherapy control board mounting hole and is opposite to the air pump motor below; a power wire is further arranged below the air pump motor; and the lamp panel is arranged around the mounting sleeve.

5. The Bluetooth speaker table lamp with essential oil aromatherapy according to claim 1, wherein the microporous tube A and the microporous tube B are arranged at 90°.

6. The Bluetooth speaker table lamp with essential oil aromatherapy according to claim 4, wherein the aromatherapy chamber is detachably connected to the essential oil bottle through threads.

7. The Bluetooth speaker table lamp with essential oil aromatherapy according to claim 1, wherein the main control board is further provided with a main control circuit, an audio power amplifier circuit, a button switch circuit, a storage circuit, an interface circuit, and a lamp panel circuit; and corresponding ports of the main control circuit are electrically connected to corresponding ports of the audio power amplifier circuit, the button switch circuit, the storage circuit, the interface circuit, and the Bluetooth module.

8. The Bluetooth speaker table lamp with essential oil aromatherapy according to claim 7, wherein the main control circuit comprises a main control chip CA51F005 and a peripheral circuit thereof.

9. The Bluetooth speaker table lamp with essential oil aromatherapy according to claim 8, wherein the audio power amplifier circuit comprises an audio power amplifier chip XS9110A and a peripheral circuit thereof; and corresponding ports of the audio power amplifier chip XS9110A are electrically connected to corresponding ports of the main control chip CA51F005.

10. The Bluetooth speaker table lamp with essential oil aromatherapy according to claim 9, wherein the Bluetooth module comprises a Bluetooth circuit, the Bluetooth circuit comprising a Bluetooth chip and a peripheral circuit thereof; and corresponding ports of the Bluetooth chip are electrically connected to the corresponding ports of the main control chip CA51F005.
